(19) United States
(12) Patent Application Publication
(10) Pub. No.: US 2010/0184132 A1
(43) Pub. Date: Jul. 22, 2010

(54) Enzymatic Process for Debittering of Protein Hydrolysate Using Immobilized Peptidases

(75) Inventors: Madhujit Vishwas Damle, Mumbai (IN); Sahayog Narayan Jamdar, Mumbai (IN); Padmanabhkurup Harikumar, Mumbai (IN)

Correspondence Address: THE WEBB LAW FIRM, P.C., 700 KOPPERS BUILDING, 436 SEVENTH AVENUE, PITTSBURGH, PA, 15219, US

(73) Assignee: SECRETARY, DEPARTMENT OF ATOMIC ENERGY, Mumbai (IN)

(21) Appl. No.: 12/160,517

(22) PCT Filed: Jan. 13, 2006
(86) PCT No.: PCT/IN2006/000025

§ 371 Date: Jan. 8, 2009

(51) Int. Cl. C12P 21/06

(52) U.S. Cl. 435/68.1

(57) ABSTRACT

A method for enzymatic debittering of protein hydrolysates comprising the steps of isolating the protein hydrolysates from animal and plant source, reacting the said protein hydrolysates with peptidases immobilized on calcium alginate beads packed in a column.

### FIELD OF INVENTION

This invention relates to a method for the enzymatic debittering of protein hydrolysates.

### BACKGROUND OF THE INVENTION

Hydrolysis of foods proteins is carried out for various reasons including improvement of nutritional characteristics, retarding deterioration, modification of functional properties such as solubility, emulsification, foaming and the removal of toxic or inhibitory ingredients.

Protein hydrolysates form an important part of medical diets for the treatment of short bowel syndrome, Crohn's disease and diets for elderly. They are also gaining acceptances as components of sports and weight control diets.

Hydrolysis of food proteins results in the production of bitter taste, which is generally, attributed to certain peptides (MW<10 kDa), rich in hydrophobic amino acids like leucine, valine, proline, phenylalanine etc. in certain sequences in the peptide.

The commonly adopted approaches for the debittering of protein hydrolysates have been
1. Removal of bitter components by solvent extraction.
2. Adsorption of bitter peptides on solid matrices.
3. Masking of bitterness by additives.
4. Treatment with peptidases.

1. Solvent, Extraction: Protein hydrolysates have been debittering by extracting the bitter principles with Secondary butyl alcohol (SBA) (Lalasidis and Sjoberg, 1978). However, it has been shown that about 50-70% of the essential amino acids from the hydrolysate are lost in the SBA fraction.

2. Adsorbents: Removal of bitter peptides by adsorbents has been patented. The list is as below
a) Roland (U.S. Pat. No. 4,075,193) using Phenolic resins.
b) Farr et. al. (U.S. Pat. No. 4.293,583) using Vegetable adsorbent.
c) Garbutt et. al (U.S. Pat. No. 5,266,685) using Amberlite resins.
d) Cordle et. al (U.S. Pat. No. 5,837,312) using Siloxanes.
   However, this approach separates the hydrophobic peptides from the hydrolysates resulting in the net loss of nutritive amino acids.

3. Additives: The additives such as polyphosphates (Tokita, 1969), glycine (Stanley, 1981), cylodextrin (Tamura et. Al, 1990) and acidic oligopeptides (Arai, 1980) have been shown to mask bitterness of protein hydrolysates. However, the incorporation of the masking agents increases the cost markedly, limiting its usefulness. Fujimaki et. al. (1970) used the plastein reaction to reduce bitterness. However, the production of toxic components accompanying plastein reaction limits its use in food applications.

4. Treatment with peptidases: Peptidases that catalyze the release of amino acids from the N and the C termini of peptides release free amino acids bringing the hydrophobic amino acids into solution. This along with the breaking of the specific combinations of amino acids in peptides leads to debittering of protein hydrolysates.

Enzymes such as Aminopeptidase T from Thermus aquaticus YT1 (Minagawa et. al., 1989), Aminopeptidase N from Lactococcus lactis sub spp cremosis WG2 (Tan et. al., 1993), Aminopeptidase from Grifofa frondosa (Basidiomycetes) Nishiwaki et. al, 2002), Aminopeptidase from Aeromonas caviae (Izawaet. Al., 1997), Carboxypeptidase from wheat (Umetsu et. al., 1983) and procine pancreatic exopeptidases (Ge and Zhang, 1996) have been used for debittering protein hydrolysates.

However the procedures mentioned above suffer from drawbacks such as low yield of enzyme (Tan et. Al, 1990; Nishiwaki et. al., 2002) and reaction times as prolonged as 20-24 h (Minagawa et. al., 1989; Izawa et. al., 1997; Nishiwaki et. al., 2002). Moreover procedures employing purified enzymes involve costly separation steps, law enzyme yields and loss of the enzyme in solution without recycling.

Kwon et. al (U.S. Pat. No. 6,214,585) have used Lactobacillus belveticus cells as a source of enzymes for debittering. However, at the end of the debittering process removal of the vegetative and spore form of the microorganisms has to be effected without which the shelf life of the product will be hampered.

Kodera et. al (U.S. Pat. No. 6,455,273) have patented a process for the producing a less bitter hydrolysate using a low specificity cysteine protease from germinating soybeans. However, the peptides produced by this enzyme have been shown to be larger than those produced by trypsin or alcalase.

The review of literature on the various debittering processes shows that the enzymatic debittering processes are better than adsorption or solvent extraction as they do not hamper the nutritive value of the hydrolysate. However, despite the immense potential of the enzymatic processes, two major constraints that hamper its application are limited availability of catalytically efficient proteases and the lack of suitable technology to recycle proteases. The patent outlined by us specifically tries to address these shortcomings as is discussed in the following sections.

### OBJECTS OF THE INVENTION

An object of this invention is to propose a method for the enzymatic debittering of protein hydrolysate.

Another object of is invention is to propose a readily available and cheap source of enzyme for the debittering method of the present invention.

Further object of this invention is to propose use of mucosal peptidases obtained by processing poultry waste.

### BRIEF DESCRIPTION OF THE INVENTION

According to this invention this provided a method for enzymatic debittering of protein hydrolysates company the steps of isolating the protein hydrolysates from animal and plant source, reacting the said protein hydrolysates in a column packed with immobilized peptidases on calcium alginate beads.

### BRIEF DESCRIPTION OF THE ACCOMPANY DRAWINGS

FIG. 1: Effect of irradiation on chicken mucosal aminopeptidases.

FIG. 2: Schematic representation of the debittering process.

FIG. 3a: RP HPLC profile of casein hydrolysate (i) and debittered casein hydrolysate (ii).

FIG. 3b: RP HPLC profile of soybean protein hydrolysate (i) and debittered soy protein hydrolysate (ii).

FIG. 4: Sensory evaluation of A) Casein and B) Soybean Hydrolysates.

### DETAILED DESCRIPTION OF THE INVENTION

A new method for the debittering of protein hydrolysates using exopeptidases associated with chicken intestinal mucosa immobilized on Calcium alginate beads. The bitter protein hydrolysate is passed over a bed of these beads packed in a column, maintained between 40° C.-60° C. and pH 5./0-8.0 and the liquid outflowing from the column is the debittered protein hydrolysate.

### Advantages

i) Chicken intestine is largely, an unused processing waste of poultry industry, which is rich in commercially important proteolytic enzymes including aminopeptidases (Jamadar et. al. 2003, Jamdar et. al. 2005,) 85% of the aminopeptidase activity of the chicken intestine is resident in the mucosal layer. Thus the enzyme source used in our method is cheap and readily available.
ii) The animal proteases are generally known to be highly efficient.
iii) Since the chicken mucosal peptidases exhibit broad specificity a wide spectrum of peptides involving almost all amino acids could be hydrolysed.
iv) More importantly the immobilization of the peptidases also does away with problem of loss of the enzyme during the process.
v) The aminopeptidases immobilized in calcium alginate beads remain fully active even after 120 days at 40° C. They lose 30% activity after 7 days at 50° C. However, in the presence of casein hydrolysate no loss of activity was observed after 7 days at 50° C., Thus, the substrate protects the enzymes from thermal inativation, which helps in longer operation of the column.
vi) Debittering is achieved by passing the hydrolysate at a flow rate of 45 ml/h through a column (43 cm×1.5 cm) packed with 30 g of beads. The pH and temperature of the system is in the range 5.0-8.0 and 40° C.-60° C. respectively.
vii) The effective output of the system could be enhanced by a scale up of the system.

The chicken intestine brought from the local abattoir is rendered free of superficial dirt, overlying fact, connective tissue and other organs (spleen, pancreas etc). The intestines are rendered free of food and faecal matter by flushing tap water through them, longitudinally cut open and the mucosal layer is scraped off. Mucosa is then packed in polythene bags and sterilized by gamma irradiation (20 kGy). The sterility of the mucosa is confirmed by the absence of growth in nutrient media inoculated with the mucosa under aerobic as well as anaerobic conditions. 70-80% of the aminopeptidase activity is retained even after irradiation (FIG. 1).

Mucosa is then mixed with 3% sodium alginate (in a proportion of 1:5 v/v) and added drop wise to a solution of CaCl2 to make Calcium alginate beads. Procedure for the immobilization of proteins in Calcium alginate is documented in literature (Smisrod and Skjak-Braek, 1990).

The column packed with beads is used to debitter protein hydrolysates.

The invention is further explained in detail with the help of the examples:

### EXAMPLE 1

A tryptic hydrolysate of casein was prepared. The concentration of this hydrolysate was about 5%. The pH of this solution was maintained between 5.0-8.0. This suspension was introduced into a column (30 g beads in a column of volume approximately 75 ml) packed with CI-Mucosal alginate beads (FIG. 2) at a flow rate of 35-50 ml hr⁻¹ (equivalent to one column void volume h⁻¹). The temperature of the column was maintained between 40-60° C. by circulating warm water through the jacked of the column. The solution emanating from the column was the debittered protein hydrolysate.

### EXAMPLE 2

Peptic hydrolysate of soybean protein (5%) was treated similar to casein hydrolysate. The pH of this solution was maintained between 5.0-8.0. This suspension was introduced into a column (30 g beads in a column of volume approximately 75 ml) packed with CI-Mucosal alginate beads (FIG. 2) at a flow rate of 35-50 ml hr⁻¹ (equivalent to one column void volume h⁻¹). The temperature of the column was maintained between 40-60° C. by circulating warm water through the jacked of the column. The solution emanating from the column was the debittered soy protein hydrolysate.

### EXAMPLE 3

### Hydrophobicity Profile

The hydrophobicity profiles of the bitter hydrolysates of casein and soybean and their debittered counterparts were analyzed on a HPLC system equipped with a RP C 18 column. The peptides were separated using a gradient from 01.% TFA(A) to 60% Acetonitrile in 0.1% TFA (B) and were monitored by absorption at 220 nm. The gradient was: 0 min-100% A, 5 min-100% A, 5 min-45 min 100-0% A, 45-50 min-0% A, 50-55 min-0-100% A, up to 65 min-100% A.

The RP HPLC profiles of casein and Soy protein hydrolysates before and after debittering are presented in FIGS. 3a and 3b respectively. It is seen that in both the cases treatment with the immobilized mucosa caused conversion of hydrophobic peptides to hydrophilic residues resulting in a distinct shift in the peptide profile of the hydrolysate towards the polar region.

### EXAMPLE 4

### Average Peptide Chain Length

The average peptide chain length of the hydrolysates (Nishiwaki et. al., 2002) before and after debittering have been calculated by estimating the free amino groups by TNBS method. (Adler-Nissen, 1979). Results show that (Table 1) the debittering is accompanied by a decrease in the average peptide chain lengths in both the cases.

### EXAMPLE 5

### Amino Acid Profile

100 mg of lyophilized hydrolysates before and after debittering were digested invacuo at 110° C., for 24 h in the presence of 6N HCl and analysed for their amino acid content after derivatization with OPA reagent. The separation of amino acids was monitored by absorbance at 330 nm. The result presented in Table 2 reveals that the debittering did not cause any change in the amino acid composition of the samples, thus, the process assures no loss on the nutritive value in terms of amino acid content.

### EXAMPLE 6

### Sensory Analysis

The organoleptic evaluation of the samples was performed by a group of taste panelists who had been selected on the basis of their sensitivity to bitterness. The scale of bitterness was formed by comparing with standard caffeine solutions.

| Caffeine Conc (%) | Scale | Taste |
|---|---|---|
| 0 | 0 | Not Bitter |
| 0.025 | 1 | Trace Bitter |
| 0.05 | 2 | Slight Bitter |
| 0.1 | 3 | Bitter |
| 0.2 | 4 | Very Bitter |
| 0.3 | 5 | Extremely Bitter |

After the treatment, the bitterness of casein hydrolysate was found to be reduced from 4.33 to 2.46 on the bitterness scale (FIG. 4a). The soybean hydrolysate scored at 3.8 while the debittered soy protein hydrolysate scored 2.43 on the bitterness scale (FIG. 4b). In both the cases debittering was also found to improve the overall acceptability of the hydrolysates.

TABLE 1
Influence of debittering on peptide chain length.

Average peptide chain length (amino acids)

| | Casein | Soybean |
|---|---|---|
| Bitter | 5.61 | 5.21 |
| Debittered | 2.59 | 2.72 |

TABLE 2
Amino Acid profile of debittered protein hydrolysates.

| Amino | Casein | Soybean Protein |
|---|---|---|
| Acid | hydrolysate | hydrolysate |
| Asp | 95.61 | 91.65 |
| Glu | 111.27 | 100.28 |
| ser | 97.25 | 86.13 |
| gly | 96.92 | 90.40 |
| thr | 96.97 | 90.87 |
| arg | 113.25 | 96.72 |
| tyr | 97.29 | 92.69 |
| val | 104.76 | 99.88 |
| met | 96.10 | 97.42 |
| ile | 105.05 | 98.35 |
| phe | 100.24 | 92.19 |
| leu | 97.07 | 99.19 |
| lys | 97.74 | 93.99 |

1-6. (canceled)

7. A method for enzymatic debittering of protein hydrolysates of plant and animal origin comprising passing the protein hydrolysates over a mixture of exopeptidases immobilized on calcium alginate beads packed in a column.

8. The method as claimed in claim 7, wherein said exopeptidases is obtained from any mucosa rich in exopeptidases.

9. The method as claimed in claim 8, wherein said mucosa rich exopeptidases is obtained from the mucosa layer of the chicken intestine.

10. The method of claim 9, further comprising:
a) cleaning the mucosa layer of the chicken intestine;
b) sterilizing the said mucosa by gamma radiation; and
c) immobilizing the mucosa in calcium alginate.

11. The method as claimed in claim 10, wherein said mucosa is mixed with 3% sodium alginate in the proportion of 1:5 v/v.

12. The method as claimed in claim 7, wherein the protein hydrolysates are passed at a flow rate of 45 ml/hr through a column containing 6 g of immobilized chicken intestinal mucosa possessing 1152 units of exopeptidase activity.

13. The method as claimed in claim 7, wherein the pH and temperature of the column is in the range of 5.0-8.0 and 40° C. to 60° C., respectively.
